(19) United States
(12) Patent Application Publication
(10) Pub. No.: US 2011/0318275 A1
(43) Pub. Date: Dec. 29, 2011

(54) MULTIFUNCTIONAL CONTRAST AGENT USING BIOCOMPATIBLE POLYMER AND PREPARATION METHOD

(75) Inventors: Sun Hang Cho, Daejeon (KR); Byung Cheol Shin, Daejeon (KR); Soon Hong Yuk, Daejeon (KR); Ha Soo Seong, Daejeon (KR); Byung Jin Kim, Daejeon (KR); Hyo Jeong Kim, Daegu (KR); Youn Woong Choi, Gyeonggi-do (KR); Byung Gu Min, Seoul (KR); Dae Chul Ha, Chungcheongnam-do (KR)

(73) Assignees: KOREA UNITED PHARM. INC., Yeongi-gun, Chungcheongnam-do (KR); KOREA RESEARCH INSTITUTE OF CHEMICAL TECHNOLOGY, Daejeon (KR)

(21) Appl. No.: 13/255,074

(22) PCT Filed: Sep. 28, 2009
(86) PCT No.: PCT/KR09/05533

§ 371 Date: Sep. 6, 2011

(30) Foreign Application Priority Data
Mar. 11, 2009 (KR) 10-2009-0020676

(51) Int. Cl. A61K 49/14; C07K 11/00; A61K 31/704; A61K 31/337; B32B 5/16; A61K 31/138; A61K 9/00; A61K 49/00; A61K 49/18; A61P 35/00; C07K 14/00; A61K 33/24

(52) U.S. Cl. 424/9.323; 530/400; 530/350; 530/300; 514/34; 514/449; 424/649; 514/651; 424/400; 424/9.322; 424/9.34; 428/402

(57) ABSTRACT

The present invention relates to a biocompatible contrast agent and a method of its preparation. More particularly, the present invention relates to a multifunctional contrast agent manufactured by prepairing a novel polysuccinimide-based polymer by introducing an alkanolamine group to the main group of the polysuccinimide in addition to a biocompatible hydrophilic group, which improves bioavailability, and a hydrophobic group, which enables to maintain the form of stable nanoparticles during the formation of nano particles for a long period of time and to encapsulate a hydrophobic anticancer agent.

### TECHNICAL FIELD

The present invention relates to a multifunctional contrast agent using a biocompatible polymer and a method of preparing the same.

### BACKGROUND ART

Of various image technologies available for early diagnosis of human diseases, MRI has been welcome as a very useful means of diagnosis and early detection of diseases due to the advantages that it is less harmful than X ray or CT because it is exposed to radiation much less, it can improve sensitivity and specificity in diagnosis, and much shortened time for acquiring images for diagnosis with the recent fast progress in MR hardware and software. MRI has been also used in place of CT for the post-treatment evaluation.

An MRI contrast agent is a colloidal solution using a paramagnetic material such as gadolinium (Gd), manganese (Mn), iron (Fe) oxide. Highly toxic materials such as gadolinium and manganese are prevented from releasing toxins by allowing them to be conjugated into a chelate comprising an organic substance(s).

However, the above has disadvantages that the chelate may be detached during injection to human body thereby incurring a risky situation for people to be exposed to toxic metals and has a very short half-life of about 14 minutes thus not being able to be used in accurate and precise diagnosis.

Meanwhile, iron oxide contrast agent is a safe contrast agent which has already passed the safety test and can stay in the body for about 8 hours thus enabling accurate diagnosis. However, the conventional iron oxide nanoparticles have not been well developed because they require a high reaction temperature of 250° C. or above, a few tens of hours of reaction time, high cost materials and a complicated manufacturing process, and thus there has been a longfelt need for the development of a novel process for their manufacture.

KR Pat. No. 634381 discloses polysuccinimide-based polymer wherein the polymer comprises polysuccinimide with a molecular weight of 1,000-100,000 as a main chain and a hydrophilic group with a molecular weight of 100-20,000, a hydrophobic group derived from C3-80 amine or phospholipid and a chelate with a contrast agent enclosed therein, and a contrast agent using the same. However, it was shown to have disadvantages of difficulty in control of particle size, resolution and sensitivity.

### DISCLOSURE OF INVENTION

### Technical Problem

As a result of continued effort for resolving the above-mentioned problems, the inventors of the present invention have solved the toxicity problem by using polysuccinimide Baypure™ DSP which was obtained by reacting the starting material with maleic anhydride. Further, the inventors also succeeded in manufacturing T2 contrast agent by introducing to the main chain of the polysuccinimide a hydrophilic group, which is biocompatible and enables to improve bioavailability, a hydrophobic group which enables to maintain the shape of the form of stable nanoparticles during the formation of nano particles, and an alkanolamine group to bind to iron oxide contrast agent.

For the purposes of diagnosis and therapeutic effect, an anticancer agent was encapsulated into the above-mentioned T2 contrast agent and theragnostic agent was manufactured thereof. Thus manufactured theragnostic agent was shown be a superior multifunction drug delivery system having improved variety, regulation of particle size, resolution, sensitivity, multifunction, therapeutic effect as compared to the contrast agent disclosed in KR Pat. No. 634381 and thereby completed the present invention.

Therefore, an objective of the present invention is to provide iron oxide contrast agent for MRI measurement for diagnosis of diseases comprising a biocompatible polysuccinimide as a main chain and various functional groups bound thereto.

Another objective of the present invention is to provide a theragnostic agent with an anticancer agent encapsulated therein for diagnosis and treatment of diseases.

A further objective of the present invention is to provide a optical imaging contrast agent for the measurement of near infrared fluorescent (NIRF).

### Solution to Problem

The present invention relates to a polysuccinimide-based compound comprising:

(a) a main chain consisting of polysuccinimide synthesized from maleic anhydride having a molecular weight in the range of 1,000-100,000; and

(b) a branch chain consisting of a hydrophilic group derived from polyethyleneglycol, polyvinylpyrrolidone, dextran, polyethyleneoxide, polylysine or polyvinylalcohol having a molecular weight in the range of 100-20,000; a hydrophobic group derived from C3-C80 amine or phospholipid; and an alkanolamine group comprising a contrast agent selected from the group consisting of gadolinium, manganese, iron oxide, aluminum, silicon, barium, yttrium and rare earth elements.

The present invention also relates to a method of manufacturing a contrast agent, a theragnostic agent, and a near infrared fluorescent contrast agent.

The present invention is described further in detail herein below.

The present invention relates to an MRI (magnetic resonance image) contrast agent using a biocompatible polymer. The contrast agent of the present invention is a multifunctional contrast agent which has an MRI contrast function due to the fact that it comprises various functional groups along with a biocompatible polysuccinimide, manufactured by using maleic anhydride as a starting material, and a drug encapsulation function.

More specifically, the polysuccinimide synthesized by using maleic anhydride as a starting material is a non-toxic polymer, which opens a ring while detaching a carboxyl group, thereby allowing introduction of various functional groups as a branch chain, and it comprises a hydrophobic group itself.

Further, the present invention relates to a method of manufacturing an MRI iron oxide contrast agent, by using a synthetic polymer, which comprises the above-mentioned polysuccinimide as a main chain, and (a) a hydrophilic group, which is well dispersed in water and improves stability and bioavailability of nanoparticles in blood, (b) a hydrophobic group, which can carry a hydrophilic group that can be easily removed from the body via filtration by kidney as well as a hydrophobic drug, and maintain the form of a micelle for a long period of time, and (c) an amine group such as alkanolamine of ethanolamine, methanolamine or propanolamine which enables dissolution of polymers by opening the ring of the carboxyl group in the main chain and the binding with a contrast agent.

The present invention also relates to a method of manufacturing a near infrared fluorescent contrast agent by introducing an alkylenediamine group instead of an iron oxide contrast agent and binding to a fluorescent material modified with N-hydroxy succinimidyl ester (NHS-ester).

The present invention also relates to a method of manufacturing a theragnostic agent by encapsulation of an anticancer agent to the above MRI iron oxide contrast agent.

In particular, the method of manufacturing a contrast agent according to the present invention is very simple which requires a much shorter time than the conventional methods, provides a high yield, and enables to control the particle size thus allowing a large scale production. Further, by encapsulation an anticancer agent in it, it can be used for diagnosis and treatment of diseases.

The manufacturing process of the present invention is divided into two steps: a step of manufacturing a synthetic polymer with various functional groups comprising polysuccinimide as a main chain for binding a contrast agent, and a step of encapsulation of an anticancer agent by using the above synthetic polymer. Each of the above steps includes steps of synthesis and purification and are described further below.

The above-mentioned synthetic polymer comprises: (a) a biocompatible and biodegradable hydrophobic polymer of polysuccinimide as a main chain, which was purchased from Baypure™ (Baypure DSP with a molecular weight of 2000-4000 g/mol); (b) a hydrophilic group with a molecular weight of 100-20000 g/mol that serves to improve in vivo stability and bioavailability; (c) a C3-80 hydrophobic group which serves to improve stability of micelles by maintaining the structures of micelles formed in an aquatic phase, and also serves to encapsulate a hydrophobic drug such as an anticancer agent via a hydrophobic interaction; (d) an alkyleneamine group such as ethanolamine which serves to open the ring of the unconjugate repeating unit parts of main chain to convert the polymer into a water-soluble polymer and also enables to bind (or conjugate) a contrast agent or an alkylenediamine group such as ethylendiamine which serves to bind the above alkyleneamine group to a near infrared fluorescent (NIRF) contrast agent.

The above-mentioned polysuccinimide used as the main chain is preferred to have a molecular weight of 1,000-100,000. If the molecular weight is less than 1,000, it may result in its diffusion to its neighboring tissues at the time of blood vessel injection. Meanwhile, if the molecular weight is greater than 100,000, it may result in overstay of the polysuccinimide in the body.

The above-mentioned polysuccinimide is a biocompatible and biodegradable poly amino acid-based polymer. Due to the presence of a functional group at each repeating unit of the polymer, it can readily bind to most functional groups, and is thus helpful to introduce various kinds of branch chains.

The above hydrophilic group is preferably at least one derived from polyethyleneglycol, polyvinylpyrrolidone, dextran, polyethyleneoxide, polyvinylalchol and polylysine, more preferably at least one derived from glycerol, propyleneglycol, ethyleneglycol, D-lysine, L-lysine, and DL-lysine.

If the molecular weight of the above hydrophilic polymer is less than 100 g/mol, the polymer will be easily decomposed thus resulting in depolymerization of micronano-sized particles. In contrast, if the molecular weight is greater than 20,000 g/mol, it will increase the toxicity in triglycerides. The hydrophilic polymer is introduced in the amount of 0.1-0.25 mol per 1 mol of polysuccinimide, the main chain. If the content of polysuccinimide is less than 0.11 mol, micronano-sized particles will not be formed. In contrast, if the content is higher than 0.25 mol, it will result in increase its toxicitiy against humans.

The above hydrophobic group is preferably at least one those derived from C3-80 amine or phospholipid. Examples of the C3-80 amine include tetradecylamine, hexadecylamine, octadecylamine and dioctadecylamine.

Phospholipid has hydrophobicity and one of the major components of cell membrane and can minimize the bodily rejection at the time of injection into the body. Its examples include hexadecylamine, albumin, liposome.

The hydrophobic group is introduced 1-5 mol per 1 mol of polysuccinimide. If the amount introduced is less than 1 mol, the nanoparticles cannot be maintained for a long period of time during the formation of nano particles. If the amount introduced is greater than 5 mol, it will incur a problem in its binding to other branch chains and extreme increase in the size of the nanoparticles.

The preferred examples of the alkanolamine group that serves to a contrast agent include ethanolamine, methanolamine, propanolamine. The amount of the alkanolamine group to be introduced is preferably 20-75 mol per 1 mol of polysuccinimide. If the amount is less than 20 mol, it will weaken its reactivity with the contrast agent thus lowering the yield. In contrast, if the amount is greater than 75 mol, it will prevent its reaction with other branch chains.

Examples of the contrast agent include gadolinium, manganese, iron oxide, aluminum, silicon, barium, yttrium and rare earth elements. For example, the process of conjugation (physical conjugation) with iron oxide is proceeded in such a manner that when iron compound is formed into Fe3O4 in basic condition, the OH group of an ethanolamine group accept electrons of Fe.

The polysuccinimide-based polymer of the present invention can be represented by the following formula 1, and is described further herein below.

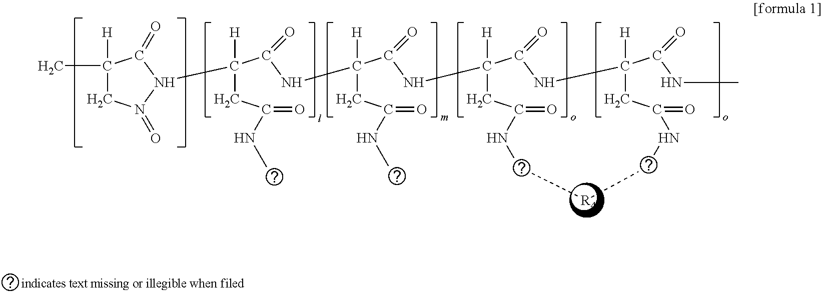

In the above formula 1, R1 represents a hydrophilic group with a molecular weight of 100-20,000 derived from polyethyleneglycol, polyvinylpyrrolidone, dextran, polyethyleneoxide, polylysine or polyvinylalcohol; R2 represents a hydrophobic group derived from C3-80 amine or phospholipid; R4 represents an alkanolamine group which conjugated (bound to) a contrast agent; R6 represents a contrast agent selected from the group consisting of gadolinium, manganese, iron oxide, aluminum, silicon, barium, yttrium and rare earth elements; l, m, o respectively represent the binding rate the hydrophilic group (R1), the hydrophobic group (R2), the alkyleneamine group (R4) to the entire number of repeating unit structure of succinimide; 1 represents 5-35 mol %, m represents 5-35 mol %, and represents 30-60 mol %.

A near infrared fluorescent contrast agent can be manufactured by introducing an alkylenediamine group, wherein a fluorescent material modified by N-hydroxy succinimidyl ester (NHS-ester) is attached to its terminal, to the above prepared polymer.

The above fluorescent material is preferably carboxyfluorescein diacetate N-succinimidyl ester, CYDye™ 3.5 (mono reactive NHS-ester) or CYDye™ 5.5 (mono reactive NHS-ester). The fluorescent material is preferably introduced in the amount of 0.0001-0.001 relative to 1 mol of polysuccinimide. If the fluorescent content is less than 0.0001 mol, the resolution at the time of near infrared fluorescent measurement deteriorates. In contrast, if the fluorescent content exceeds 0.001 mol it results in increase of the product particles.

The above alkylenediamine group is preferably methylenediamine, ethylendiamine or propylenediamine. The above alkylenediamine group is introduced 3-15 mol relative to 1 mol of polysuccinimide, the main chain. If the content of the alkylenediamine group is greater than 3 mol, it results in decrease in reactivity with other branch chains to be introduced. In contrast, if it exceeds 15 mol, it becomes a strong cation thus releasing toxicity.

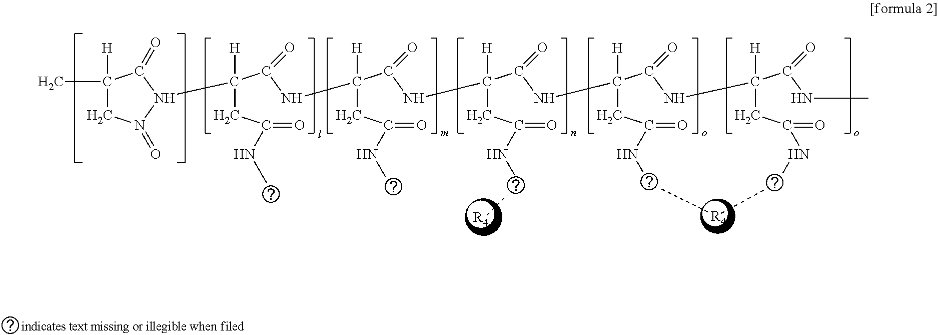

In the above formula 2, R1 represents a hydrophilic group with a molecular weight of 100-20,000 derived from polyethyleneglycol, polyvinylpyrrolidone, dextran, polyethyleneoxide, polylysine or polyvinylalcohol; R2 represents a hydrophobic group derived from C3-80 amine or phospholipid; R3 represents an alkylenediamine group; R4 represents an alkanolamine group which conjugated a contrast agent (a chelator group); R5 represents a fluorescent group modified with N-hydroxy succinimidyl ester (NHS-ester); R6 represents a contrast agent selected from the group consisting of gadolinium, manganese, iron oxide, aluminum, silicon, barium, yttrium and rare earth elements; l, m, n, o respectively represents the binding rate the hydrophilic group (R1), the hydrophobic group (R2), the alkylenediamine group (R3), the alkyleneamine group (chelator group) (R4) to the entire number of repeating unit structure of succinimide; 1 represents 5-35 mol %, m represents 5-35 mol %, n represents 5-15 mol %, and o represents 30-60 mol %.

Meanwhile, a near infrared fluorescent contrast agent can be manufactured by introducing an alkylenediamine group, wherein a fluorescent material modified with N-hydroxy succinimidyl ester (NHS-ester) is attached to its terminal, to the above prepared polymer.

Thus prepared synthetic polymer by polymerization between the main chain of polysuccinimide and the introduced materials mentioned above are reacted at 50-100° C. by using a solvent such as dimethylformimide (DMF) or dimethylsulfoxide (DMSO). Upon completion of the reaction, unreacted polymers and impurities are removed by using ethylether, and dried under vacuum dry pump to obtain purified powdered synthetic polymer.

Further, in the present invention, the above polysuccinimide-based polymer and a contrast agent are synthesized, purified and then a multifunctional contrast agent which can encapsulate an anticancer agent is prepared.

Examples of the above anticancer agent are doxorubicin, epirubicin, docetaxel, paclitaxel, valrubicin, cisplatin and tamoxifen.

In the present invention, in particular, a mixed solution where a contrast agent is dissolved in dimethylsulfoxide or dimethylformimide and an anticancer agent are irradiated by using an ultra sonicator for 100 minutes or less thereby manufacturing a multifunctional contrast agent containing an anticancer agent.

A contrast agent with a hydrophilic and a hydrophobic group is not dissolved in an organic solvent to be particulated but remains as a polymer chain as bound, wherein an anticancer agent with hydrophobic property receives a kinetic energy and is encapsulated via hydrophobic interaction as well as self-assembly in the aqueous phase.

The multifunctional contrast agent of the present invention, by introduction of various functional groups into branch chains of the polysuccinimide, enables perform multiple functions concurrently including biodegradation, minimizing bodily rejection, increase of bioavailability, increase of efficacies of contrast agent, drug loading, etc.

Of the various contrast agents using biocompatible polymers, the poly amino acid-based polysuccinimide, used as the main chain, of the present invention has advantageous features that it has excellent reactivity with other compounds thus allowing easy introduction of branch chains, has no toxicity, and the time for its biodegradation can be easily regulated.

The contrast agent of the present invention, as compared to the conventional ones, is capable of introducing a fluorescent material, an MRI contrast agent, and an anticancer agent, thus providing variety, and multifunctions. Further, as shown in FIG. 15, its particle size can be adjusted and also the contrast lymph nodes and blood vessels are possible, thus showing that it contributes greatly in terms of resolution and sensitivity. In addition, the contrast agent of the present invention, with improved functions, can provide both the contrasting effect and anticancer effects at the same time.

### Advantageous Effects of Invention

The present invention relates to a polysuccinimide-based polymer with a particle size of 20-200 nm, which is biodegradable, minimizes bodily rejection, increases bioavailability, efficacies of MRI iron oxide contrast agent or a near infrared fluorescent contrast agent, enables encapsulation of an anticancer agent, by introducing various functional groups as branch chains of the polysuccinimide, and a contrast agent comprising the same.

Therefore, the contrast agent of the present invention comprising the polysuccinimide-based polymer can be used as a theragnostic agent for diagnosis and therapeutic agent and is also expected to be used in various medical fields.

### EXAMPLES

The present invention is described further in detail with reference to the following examples but they should not be construed as limiting the scope of the same.

### Example 1

### Synthesis of Polysuccinimide-Based Polymer

1) Introduction of Branch Chain of Polyethyleneglycol to Polysuccinimide (Introduction of a Hydrophilic Group)

As for the polysuccinimide used as the main chain, Baypure DSP (Baypure™) with a molecular weight of 2,000-4,000 g/mol was purchased. 0.0025 mol of the polysuccinimide was dissolved in DMF under nitrogen atmosphere. While stirring the mixture, 0.0006 mol of polyethyleneglycolamine (5,000 g/mol) dissolved in DMF was slowly dropped thereto, and allowed to react at 60° C. for about 24 hours. The resultant was cooled down at room temperature for about 1 hour and then dropwisely added into ethylether to obtain a brown precipitate. The precipitate was passed through a membrane filter paper and then the solvent in the precipitate was removed under vacuum to obtain a fine powdered compound. Thus obtained product was analyzed by 1H-NMR and the result is shown in FIG. 1. The result shows that polyethyleneglycol is introduced as a branch chain to the polysuccinimide.

2) Introduction of Branch Chains of Hexadecylamine and Ethanolamine (Introduction of a Hydrophobic Group and an Alkanolamine Group)

0.001 mol of the compound synthesized in 1) above was dissolved in DMF under nitrogen atmosphere. While stirring the mixture, 0.0067 mol of hexadecylamine dissolved in DMF was dropwisely added thereto. The two solutions were reacted by stirring them at 60° C. for 24 hours. Upon completion of the reaction, the resultant was cooled down for about an hour, slowly added with ethanolamine 8 mL (0.133 mol) and then stirred at room temperature for 24 hours. The resultant was dropwisely added to ethylether and obtained a precipitate. Thus obtained brown precipitate was passed through a membrane filter paper and then the solvent in the precipitate was removed under vacuum to obtain a fine powdered compound. Thus obtained product was analyzed by 1H-NMR and the result is shown in FIGS. 2 and 3. The result shows that hexadecylamine and ethanolamine are introduced as a branch chain to the polysuccinimide. Likewise, tetradecylamine, octadecylamine, dioctadecylamine and phospholipid were also able to be introduced as a branch chain.

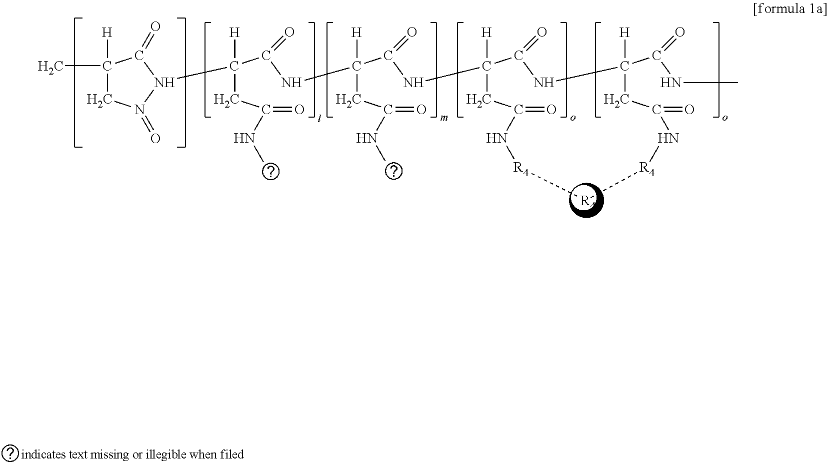

In the above formula 1a; R1 represents PEG; R2 represents hexadecylamine; R4 represents ethanolamine; R6 represents iron oxide; and l, m, o respectively represent the binding rate of the hydrophilic group (R1), the hydrophobic group (R2), the alkanolamine group (a chelator group) (R4) to the entire number of repeating unit structure of succinimide, wherein 1 represents 5-35 mol %, m represents 5-35 mol %, and o represents 30-60 mol %.

3) Introduction of Branch Chains of Alkylenediamine

Solution B, prepared by mixing 10 mL of 0.015 mol ethylendiamine with 15 mL of DMF, was slowly added while stirring to solution A, prepared by dissolving 0.0005 mol of the compound synthesized in 2) above in 15 mL of DMF at 60° C. under nitrogen atmosphere. After 6 hours of reaction between the two solutions, the resultant was removed of its remaining solvent as much as possible, dissolved in 10 Ml of deionized water, removed of unreacted monomers by using a dialysis membrane (MW 3500) for 48 hours. Upon completion of the dialysis followed by lyophilization, there was obtained a fine powdered compound.

Ethylenediamine (ED) was introduced to a polymer prepared in Example 1-2), and the portion of the PSI repeating group which was not introduced with a branch group underwent a ring opening reaction by using ethylenediamine to obtain poly hydroxyethyl aspartamide (PHEA). Thus obtained polymer was PHEA-mPEG-C16-ED which is well dispersed in aquatic phase. Thus obtained branch polymer was confirmed of the presence of polymerization with covalently bonded ethylenediamine by means of methylene peaks at around 1.3, 2.4 ppm via 1H-NMR, as shown in FIG. 4.

The polymer was identified as PSI-mPEG-C16-ED a synthetic polymer having a weight average molecular weight of 27,000 by GPC analysis. The yield was about 82% calculated by quantitation after 48 hours of dialysis followed by lyophilization.

10 mg of the polymerized polymer of PHEA-mPEG-C16-ED was dispersed in tertiary distilled water and the size and surface electric potential of the micelle particles were measured by using electrophoretic light scattering spectrophotometer (ELS-8000. Otsuka Electronics, Japan). The surface electric potential was measured at 25° C., pH 7. TEM (Transmission Electron Microscope, JEM-2010, JEOL) was used to identify the size and shape of the particles. TEM specimen was dispersed in tertiary distilled water, added with a drop of carbon-coated copper lattice of 300 mesh, and dried to be measured.

FIG. 5 confirms that, in case PHEA-mPEG-C16-ED branched polymer formed micelles in a 1 wt % aqueous environment, it(PHEA-mPEG-C16-ED micelles) has spherical particles via 20 nm and 50 nm scale bar TEM images, and also that its average particle size is about 23 nm, and laser distribution of particle size with average particle size of 10.6±6.7 nm. Based on these, it was confirmed that spherical micelles were able to be prepared.

Thus obtained powdered product was dispersed in deionized water, and measured by using a zeta-potential measuring device, and the result is shown in FIG. 6. Based on the change of zeta potential from negative (−) to positive (+) it was confirmed that ethylendiamine with a positive (+) zeta potential was introduced.

Likewise, methylenediamine and propylenediamine, wherein an amine group is present in both terminals, were able to be introduced as a main chain.

4) Introduction of a Fluorescent Material

The synthetic product in 3) above was used to manufacture nanoparticles bound to a fluorescent material by binding it with a fluorescent material such as carboxyfluorescein diacetate N-succinimidyl ester, CY 3.5 NHS-ester, CY 5.5 NHS-ester modified with NHS-ester.

CY 5.5 NHS-ester has the chemical structure 3 shown below. NHS-ester group is a functional group, which is most convenient for labelling of peptides and is thus frequently used. The most useful reaction for labelling of an amino group is acylation.

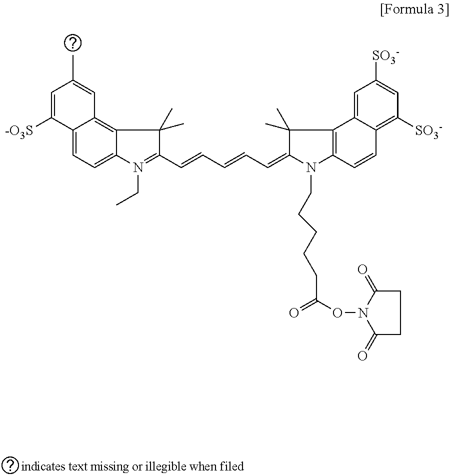

Most protein labelling is performed in phosphate, bicarbonate/carbonate and borate buffers at pH 7-9 [G. H. Haggis, D. Michie, A. R. Muir, K. B. Roberts, P. M. B. Walker, Longmans (Bristol) Green & Co. LTD., (1965)]. Based on this, the fluorescent material was introduced as in the method of Veiseh et al.

0.00083 mol of the synthetic product in 3) above and 1.3 nmol of CY 5.5 NHS-ester were respectively dispersed or dissolved in bicarbonate buffer (pH 8.5) slowly added with an aqueous solution of CY 5.5 NHS-ester (dissolved in DMF) and allowed to react at room temperature.

The resultant was removed of unreacted CY 5.5 and NHS-ester group by using a dialysis membrane (MWCO 3500, Viskase Sales Inc., Chicago, Ill., U.S.A.) in a dark room kept at 4° C. for 48 hours in PBS (phosphate-buffered saline, pH 7.4), and finally obtained PHEA-mPEG-C16-ED-Cy5.5.

The product obtained by introduction of a fluorescent material into PHEA-mPEG-C16-ED, followed by dialysis, underwent a serial dilution of 20% in PBS as shown in FIG. 7, and the presence of fluorescent material was confirmed via visual assessment. Also, with regard to the formation and size of particles, TEM image in FIG. 8 confirms that there were formed spherical shape of particles with about 3-8 nm in diameter.

From the above, it was confirmed that fluorescent material was introduced as a branch group to polysuccinimide.

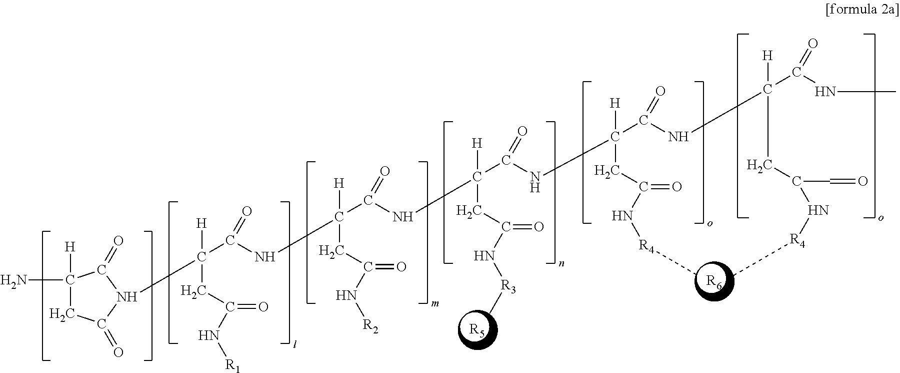

In the above formula 2a; R1 represents PEG; R2 represents hexadecylamine; R3 represents ethylendiamine; R4 represents ethanolamine; R5 represents CY 5.5 NHS-ester; R6 represents iron oxide; and l, m, n, and o respectively represent the binding rate of the hydrophilic group (R1), the hydrophobic group (R2), the alkylenediamine group (R3), the alkanolamine group (a chelator group) (R4) to the entire number of repeating unit structure of succinimide, wherein 1 represents 5-35 mol %, m represents 5-35 mol %, n represents 5-15 mol %, and o represents 30-60 mol %.

Quantitative analysis of fluorescent materials remaining in the final sample after purification was performed by using UV/visible light spectrophotometer (Shimadzu UV mini 1240, Japan) and calculated by the Lambert-Beer law equation shown below [G. Giammona, G. Puglisi, G. Cavallaro, A. Spadaro, G. Pitarresi, J. Control. Release., 33, 261-271, (1995)].

C=A/(kd) [Equation 1]

A represents optical density obtained by measurement of 1 mL of a sample at maximum absorption wavelength, k represents optical density coefficient which is 45,000 at maximum absorption wavelength, and d represents the length of passage of 1 cm, through which light is transmitted in a quartz cell, at the time of using UV/visible light spectrophotometer, and represents the value of ‘1’.

The initial concentration of the product was diluted 20 fold in PBS and the maximum absorption wavelength was measured. Free CY 5.5 dye is known to have the maximum absorption wavelength at 673 nm [S. R. Mujumdar, R. B. Mujumdar, C. M. Grant, A. S. Waggoner, Biocon. Chem., 7, 356-362, (1996)].

As shown in FIG. 9, PHEA-mPEG-C16-ED-CY 5.5 was shown to have the maximum absorption wavelength at 678 nm. The result of quantitative analysis of the amount of CY 5.5 introduced showed that about 0.928 μg of CY 5.5 is contained in the final sample solution.

The following Table 1 shows the physical properties of a near infrared fluorescent contrast agent in terms of particle size via ELS, measurement of particle surface electric potential via zeta potential, loading efficiency via a UV-vis spectromer.

TABLE 1
Near infrared fluorescent contrast agent data using polymers of Example 1

| Loading | Particle size | Zeta potential | Loading |
|---|---|---|---|
| amount (mol) | distribution (nm) | range (mv) | Efficiency (%) |
| 0.000000444 | 55-75 | (−5)-(−10) | 70-80 |
| 0.000000887 | 55-75 | (−8)-(−15) | 70-80 |
| 0.0000017 | 80-120 | (−8)-(−15) | 60-70 |

### Example 2

### Synthesis of a Contrast Agent

A purified contrast agent with introduced branch chains can be obtained via a two step process.

1) Synthesis of a Contrast Agent

0.00073 mol of the polysuccinimide-based polymer prepared in the above Example 1-2), 0.0033 mol of FeCl2.4H2O (ferrous chloride tetrahydrate) and 0.0049 mol of FeCl3.6H2O (ferric chloride hexahydrate) were added into 200 mL of tertiary distilled water, which was filled with nitrogen gas, completely dissolved, and then stirred by using a mechanical stirrer under nitrogen atmosphere while slowly adding with 30 mL of ammonia solution using a syringe pump (Kd scientific, USA). Here, the syringe used was with a 20G needle, and the injection was performed at the rate of 6 mL/h from the beginning till 20 minutes, and 7 mL/h thereafter. The stirring was vigorously performed but not to generate any foams of the mixed solution, and the reaction was performed for a total of 1 hour including the time required for injecting the ammonia water. Upon completion of the reaction, the reaction solution was cooled down at room temperature for about 30 minutes, added to 600 mL of tertiary distilled water, and then mixed to remove unreacted polymers. Here, the reaction solution before the mixing with the tertiary distilled water had strong alkalinity with a pH 10-13.

2) Purification of a Contrast Agent

The purification of the contrast agent produced above is performed through a two step process: (1) removing of impurities and then contrast agent with large particle size; and (2) removing unreacted polymers and adjusting the alkaline contrast agent in the range of pH 6.5-7.0.

In the first step, membrane filter paper (0.2 μm) was inserted into a reduced pressure flask with aspirator device and purification was performed using a vacuum pump. In the second step, unreacted polymers were removed by using a device “Dialysis with Tangential flow separation module (Hallow fiber filter membrane)” and then adjusted the pH to 6-7. Here, the media/rating was PS/50 kD and about 5 L of tertiary distilled water was used. The purification using the above device is much more efficient and effective than when performing by means of dialysis membrane tubing in terms of time and cost involved therein.

As a result of synthesis of a contrast agent, its purification followed by lyophilization, there was obtained a contrast agent in brown powder. Thus obtained final contrast agent was confirmed of its particle size and shape via TEM and the results are shown in FIG. 10. The result revealed that the contrast agent had a size of about 30 nm and are spherical at the time of ELS measurement. Further, it was shown that thus produced contrast agent has about 9.8% of iron content and is superparamagnetic. By element analysis via XPS, it was revealed that thus produced contrast agent contains a little amount of iron on the surface because of the coating by a synthetic polymer.

In addition, for the confirmation of its contrast effect, MRI was performed in vitro after manufacturing a phantom and Resovist as a control. When the concentration was made same as that of a control, it showed an effect equal or slightly better than that of the control.

For in vivo experiment, the above contrast agent in the amount of 40 μg·mol Fe/kg was injected into the ear vein of a rabbit (3 kg) with VX2 liver cancer, and the contrast effect in the liver was examined by MRI from the time prior to 0 minutes (immediately) to 20 minutes after the injection. The result is shown in FIG. 11 and the normal tissues and the lesion tissues (cancer) were distinguishable.

Further, the above contrast agent with a particle size of about 30 nm is a USPIO (Ultra small Superparamagnetic Iron Oxide), which, due to its small particle size, can circulate in the blood for a long period of time, adsorbed to particular regions as well as small regions, thereby providing contrast effect.

The above contrast agent in the amount of 80 μg·mol Fe/kg was injected into the tail vein of a rat (300 g) and then contrast effect in the aorta and great vena cava was examined by means of TI technic using an MRI, and the result is shown in FIG. 12. In 36 hours, the contrast effect in lymph nodes was examined by means of T2 technic and their tissues were pathologically examined. The results are shown in FIGS. 13 and 14, and they confirmed that the above contrast agent is capable of providing contrast effect in blood vessels and lymph nodes as well as in liver.

### Example 3

### Distribution in Particle Size According to Change in Composition and Content of Synthetic Polymer and Amount of Iron Compound

As another feature of the above MRI iron oxide contrast agent in Example 2, the MRI iron oxide contrast agent, produced as a result of the synthesis rate of hexadecylamine as a hydrophobic polymer to the polysuccinimide as a main chain, and the reaction between the synthetic polymer and iron compound according to their respective amount, was able to reproduce particles with a various size.

A contrast agent with various particle size of about 20-150 nm was prepared according to the reaction conditions and formation described in FIG. 15 by using a method same as in Example 2.

Synthetic polymers prepared by mixing hexadecylamine (a hydrophobic polymer) with polysuccinimide (the main chain) in a synthesis ratio of 15%, 25%, 35%, respectively, were reacted with an iron compound solute in a weight ratio of 4:3, 4:15, 4:1, 4:0.75, and obtained a contrast agent having the above-mentioned particle size of about 20-150 nm. Here, the value of the iron compound is the mol value of the iron compound mentioned in Example 2.

The contrast agent with various particle size has the magnetization in the range of 20-80 emu/g Fe of saturated magnetic density, and showed to have an iron content of about 5-12%.

The synthesis process of manufacturing the above contrast agent is a very rapid and vehement process and is also sensitive and thus may be limited to some extent depending on the experimental conditions mentioned above.

### Example 4

### Anticancer Agent Loading into a Contrast Agent

The contrast agent prepared in the above Example 2 with a particle size of about 30 nm can be load with anticancer agent such as Doxorubicin, Epirubicin, etc.

Sample A was prepared by mixing 100 mg of a contrast agent with 3 mL of dimethylsolfonoxide (DMSO), and sample B was prepared by dissolving 15 mg of Doxorubicin-HCl in 1 mL of DMSO, added with 150 μl of triethanolamine, and to obtain Doxorubicin or Epirubicin with hydrophobicity by deprotonation of acid.

The mixing process of sample A and reaction B were introduced with irradiation by using a sonication bath for 5 minutes and 1 minute, respectively.

While irradiating using an ultrasonicator, the sample C, which was prepared by irradiating a mixture solution of samples A and B for 2 minutes using an ultrasonicator, was dispersed in sample D containing 40 mL of tertiary distilled water by using a 26G syringe. Here, the temperature of the solution was kept at 10° C. or below by using an ice bath, and it was irradiated with the output power of 50 W for 10 minutes by using the ultrasonicator, and treated at the injection rate of 200 μl/min for 1 minute and then left therein for 30 seconds. The entire process was repeated 5 times.

Upon completion of the above process, the final mixed solution was added into a dialysis membrane tubing (MW 10,000) and dialyzed for 48 hours in a 4° C. refrigerator thereby removing unloaded anticancer agents and reactants.

After 48 hours of dialysis, the resultant was lyophilized to obtain a theragnostic agent in powder (FIG. 16).

Thus obtained the theragnostic agent was shown to have an excellent dispersion in an aquatic phase, have a particle size of about 60-70 nm, a particle surface electric potential of −20˜-30 mV, and the loading efficiency of anticancer agents measured by using UV-Vis was about 80-90%.

The following Table 2 shows physical properties of the theragnostic agent in terms of particle size via ELS, particle surface electric potential and loading efficiency measured by using UV-Vis.

TABLE 2
The theragnostic agent data obtained by using
the contrast agent of Example 4

| Loading | Particle size | Zeta potential | Loading |
|---|---|---|---|
| amount (%) | distribution (nm) | range (mv) | Efficiency (%) |
| 5 | 60-70 | 20-30 | 85-90 |
| 10 | 60-70 | 20-30 | 85-90 |
| 15 | 60-70 | 20-30 | 85-90 |
| 20 | 150-200 | 20-30 | 70-80 |

FIG. 17 shows the data confirming the anticancer effect of the theragnostic agent by its intravenous injection in experimental mice, which were allografted with B16F10 murine melanoma cell.

FIG. 18 confirms the anticancer effect of the theragnostic agent by using the ablated cancer cells.

FIG. 19 confirms the anticancer effect of the theragnostic agent by MRI image obtained according to time passage after its' intravenous injection into experimental rabbits which were induced with cancer by subculturing a VX2 hepatoma cell.

### Example 5

### In Vivo Near-Infrared Fluorescent (NIRF) Imaging Measurement

Six-week old nude mice (Female) were used for the measurement of infrared images, with a week of adaptation period after they were fasted 12 hours before the onset of the experiment.

A total of five mice were anesthetized with 1.5% isofluorane. Luminescence and flourescence animal imaging system (Xenogen corporation, KBSI-chunchen center, Korea) was used to obtain fluorescent images. The biofluorescence released from the mice was used to obtain images by using a high sensitivity charge-coupled device (CCD) camera prior to sample injection. To obtain the fluorescent images, excitation passband-filter at 615-665 nm was used, and excitation passband-filter at 695-770 nm was used and then exposed for 0.1 second. Photon flow concentration was expressed and calculated by IVIS image device program in terms of photon/second/cm2p.

100 ul of the sample solution PHEA-mPEG-C16-ED-CY 5.5 was injected through veins, and images before the injection, and 0.5, 1, 3, 6, 9, 12, 24 and 48 hours after the injection were obtained. After obtaining the image 24 hours after the injection, organs of liver, lung, heart, kidney, spleen, bladder and brain were ablated and analyzed by means of infrared images.

100 ul of the sample solution PHEA-mPEG-C16-ED-CY 5.5 contained about 0.428 uM of a fluorescent material. No biofluorescence was detected from the mice fasted 12 hours before the experiment.

After sample injection, fluorescent images reached sufficient saturation from 30 minutes after the injection even with 0.1 second of exposure. Further, it was confirmed that the sample particles containing CY 5.5 were circulated for a long period of time and also accumulated based on the images obtained until 48 hours after the injection.

FIG. 20 shows biofluorescence image (A) before the sample injection, and the fluorescent images until 24 hours after the injection (B).

The sample particles of PHEA-mPEG-C16-ED-CY 5.5 was circulated around liver, kidney, lung and bladder and was confirmed that it was accumulated. It is speculated that the sample was circulated according to time passage to various organs and accumulated because the sample particles are hydrophilic and have a small size.

FIG. 21 shows the images obtained 24 hours after the sample injection after organ ablation, and it is shown that the sample particles containing CY 5.5 were mostly accumulated in liver based on the highest fluorescence. In kidney, the central part showed a low fluorescence rate while the outer part showed a relatively higher fluorescence rate, thus suggesting the filtration or release of fluorescent molecule. Meanwhile, the sample particles were detected with low fluorescence in brain, and also detected at the time of ablation 48 hours the injection, and thus it is expected to be useful as brain contrast agent.

### BRIEF DESCRIPTION OF DRAWINGS

FIG. 1 shows graphs of 1H-NMR result of polyethyleneglycol introduced to the branch chain of polysuccinimide prepared in Example 1-1) according to an embodiment of the present invention.

FIG. 2 shows graphs of 1H-NMR result of hexadecylamine introduced to the branch chain of polysuccinimide prepared in Example 1-2) according to an embodiment of the present invention.

FIG. 3 shows a graph of 1H-NMR result of ethanolamine introduced to the branch chain of polysuccinimide prepared in Example 1-2) according to an embodiment of the present invention.

FIG. 4 shows a graph of 1H-NMR result of ethylenediamine introduced to the branch chain of polysuccinimide prepared in Example 1-3) according to an embodiment of the present invention.

FIG. 5 are graphs shows TEM image (1) and laser distribution (2) of polysuccinimide prepared in Example 1-3) according to an embodiment of the present invention.

FIG. 6 shows graphs showing the zeta potential of Examples 1-2) (left) and Example 1-3) (right) for the identification of ethylendiamine introduced to the branch chain of polysuccinimide prepared in Example 1-3) according to an embodiment of the present invention.

FIG. 7 is a picture of the product after purification for the identification of a fluorescent material introduced to the branch chain of polysuccinimide prepared in Example 1-4) according to an embodiment of the present invention (A sequential 20% serial dilution in the direction of from right to left of the initial concentration at right.

FIG. 8 is a picture showing the presence of particle formation via TEM of a fluorescent material introduced to the branch chain of polysuccinimide prepared in Example 1-4) according to an embodiment of the present invention.

FIG. 9 is a graph showing the maximum absorption wavelength of polysuccinimide prepared in Example 1-4) according to an embodiment of the present invention.

FIG. 10 shows the shape, diameter and size distribution of the particle via TEM and ELS of a contrast agent prepared in Example 2 according to an embodiment of the present invention.

FIG. 11 shows MRI images of a rabbit as a VX2 liver cancer model for in vivo experiment to study the contrast effect of a contrast agent prepared in Example 2 according to an embodiment of the present invention.

FIG. 12 shows MRI images of the aorta and vena cava of a rat to study the angiography of a contrast agent prepared in Example 2 according to an embodiment of the present invention.

FIG. 13 shows MRI images of the lymph node in the femoral region of a rat to study the lymph node contrast effect of a contrast agent prepared in Example 2 according to an embodiment of the present invention.

FIG. 14 shows pictures of histological result of the lymph node region of the above FIG. 8 dyed with Prussian Blue.

FIG. 15 shows the distribution in particle size of the MRI iron oxide contrast agent according to the composition and content of a synthetic polymer and iron compound prepared according to a method in Example 2 of the present invention.

FIG. 16 is a picture showing the physical properties of a contrast agent, wherein an anticancer agent is loaded, in an aqueous phase and in powder form.

FIG. 17 shows the change in tumor size of experimental mice to study the anticancer activity of the theragnostic agent prepared using a method in Example 4 of the present invention according to time passage.

FIG. 18 shows the pictures of ablated tumors of experimental mice (mouse) to study the anticancer activity of the theragnostic agent prepared using a method in Example 4 of the present invention.

FIG. 19 shows the change in tumor size of experimental rabbit to study the anticancer activity of the theragnostic agent prepared using a method in Example 4 of the present invention according to time passage.

FIG. 20 (A) shows biofluorescence image of nude mice obtained by using a high sensitivity CCD camera before sample injection; and FIG. 20 (B) shows a fluorescent image of polysuccinimide polymer solution prepared in Example 1-4) according to an embodiment of the present invention 24 hours after intra venous injection (a: liver, b: kidney, c: lung).

FIG. 21 shows images obtained 24 hours after intra venous injection of polysuccinimide polymer solution prepared in Example 1-4) according to an embodiment of the present invention [(A) a picture image after ablation, (B) an optical fluorescent image, a: heart, b: lung, c: bladder, d: liver, e: spleen, f: kidney, g: brain].

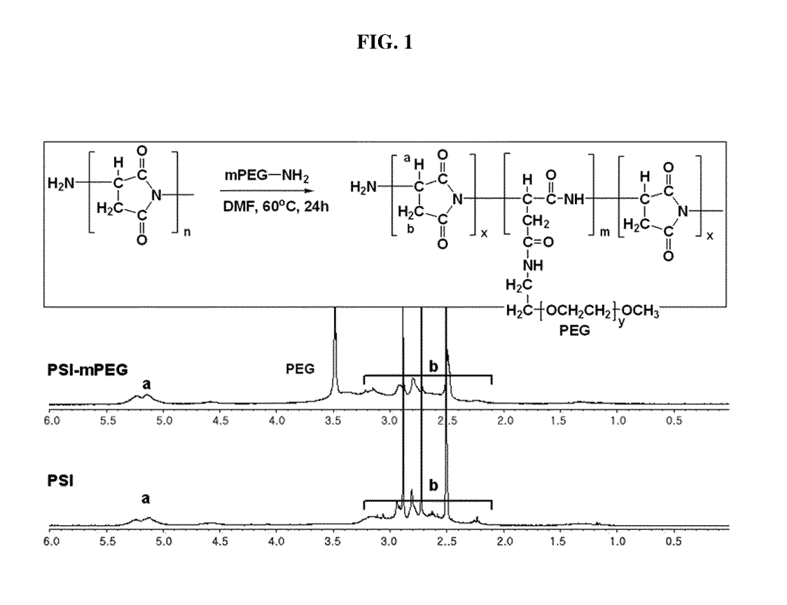

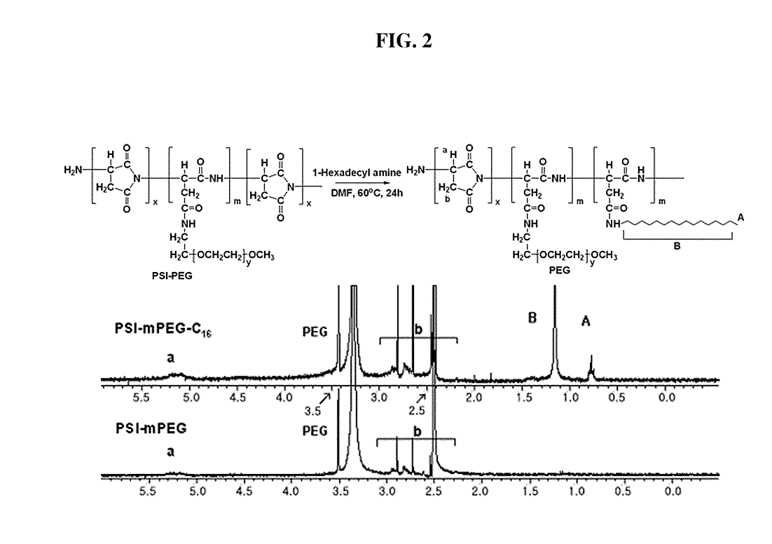

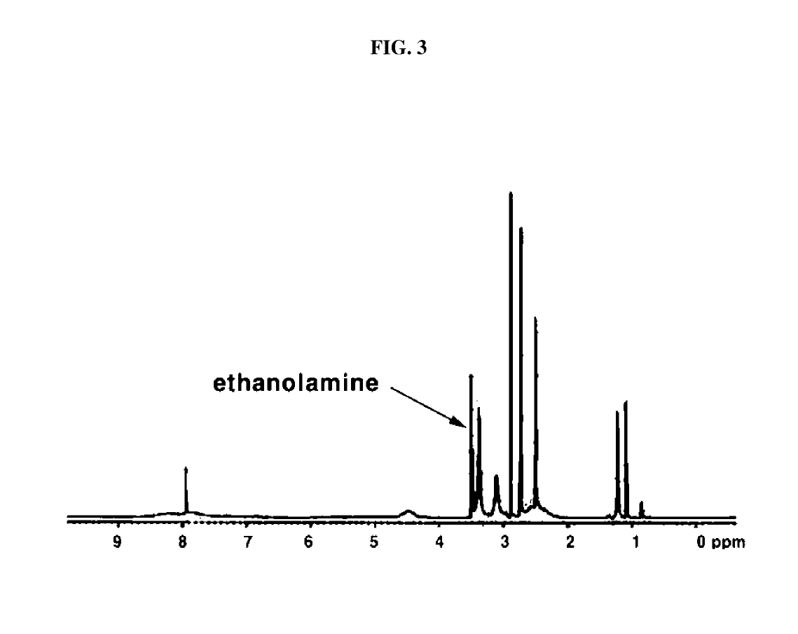

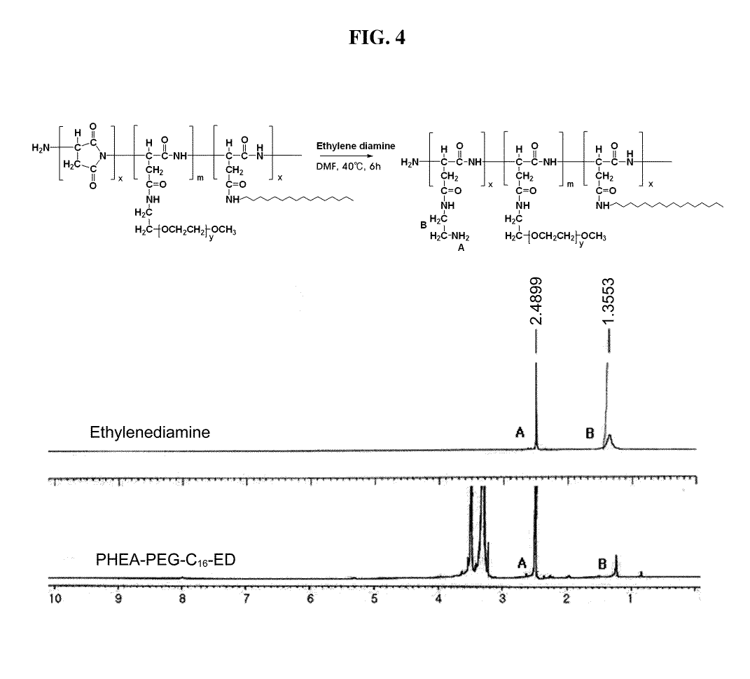

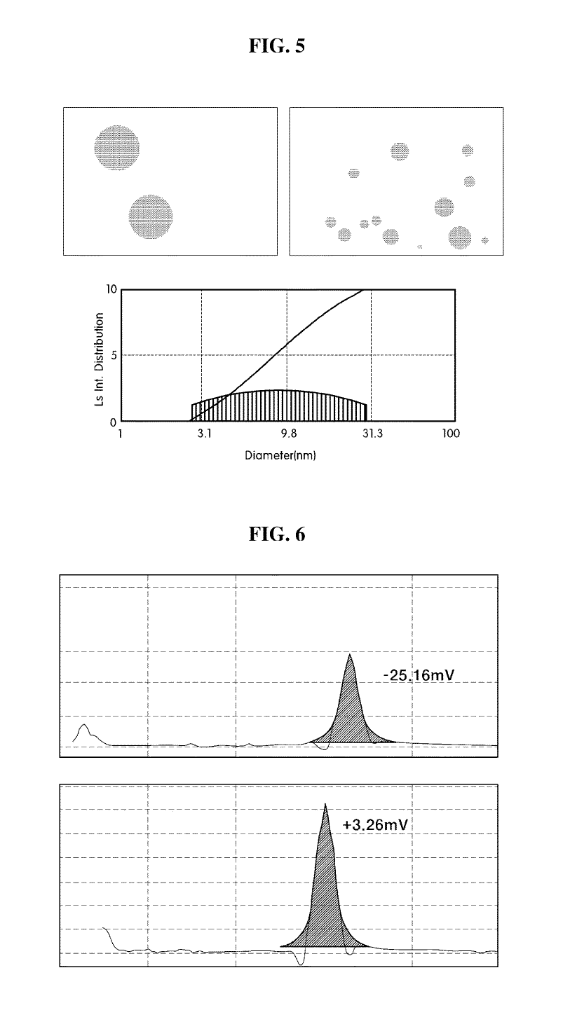

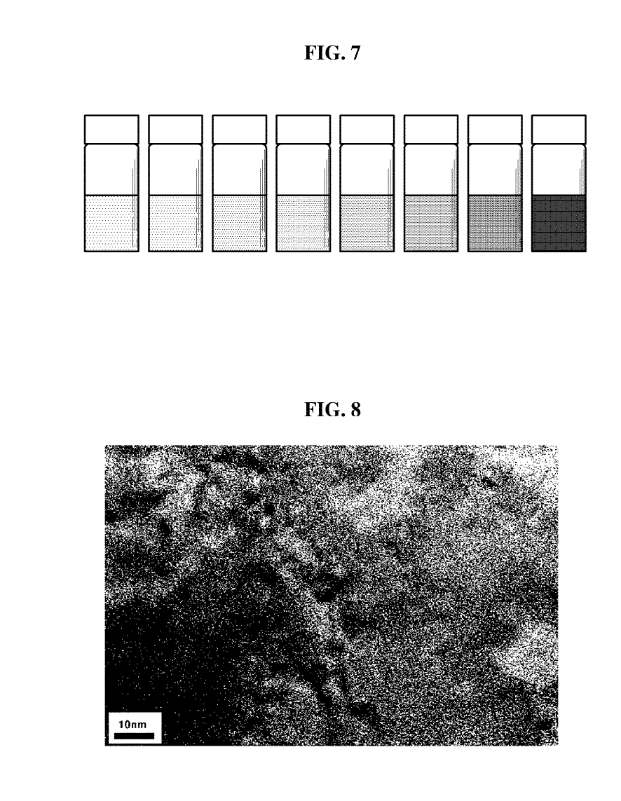

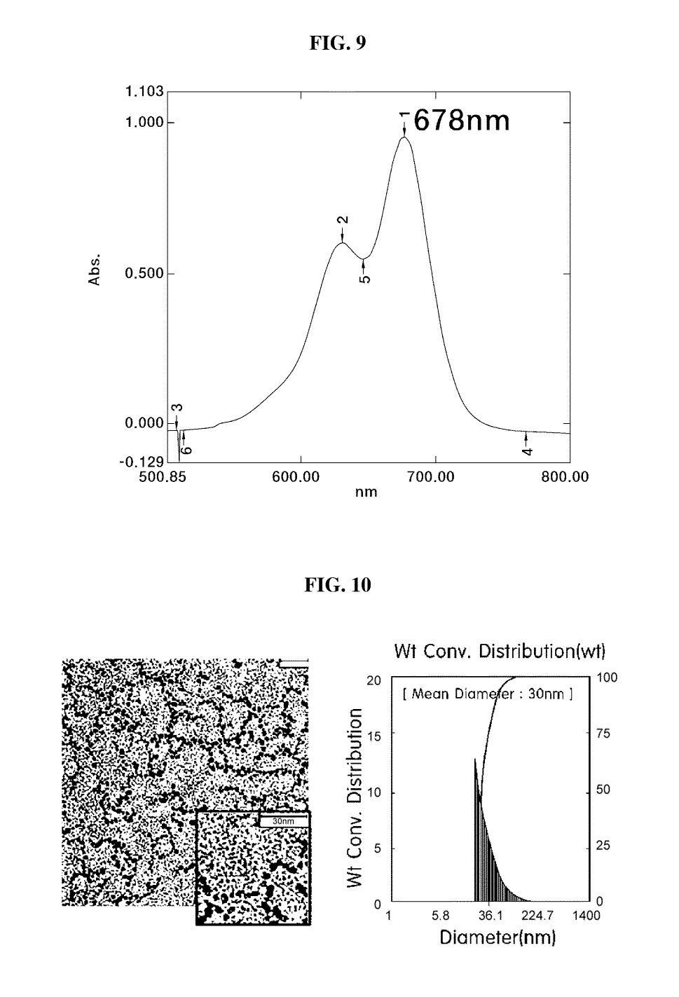

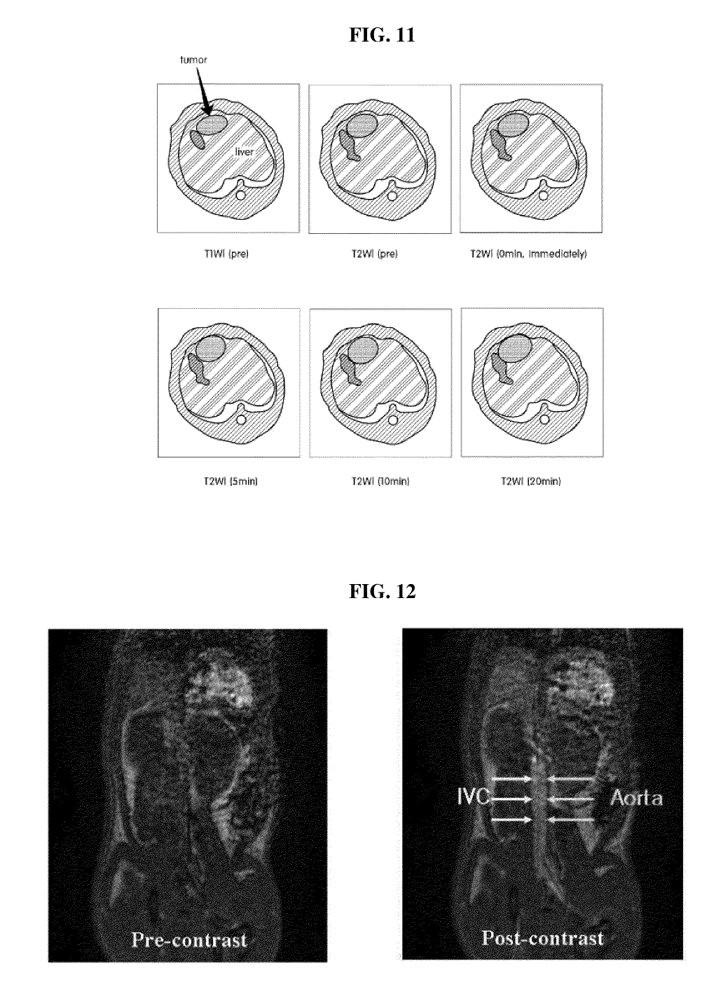

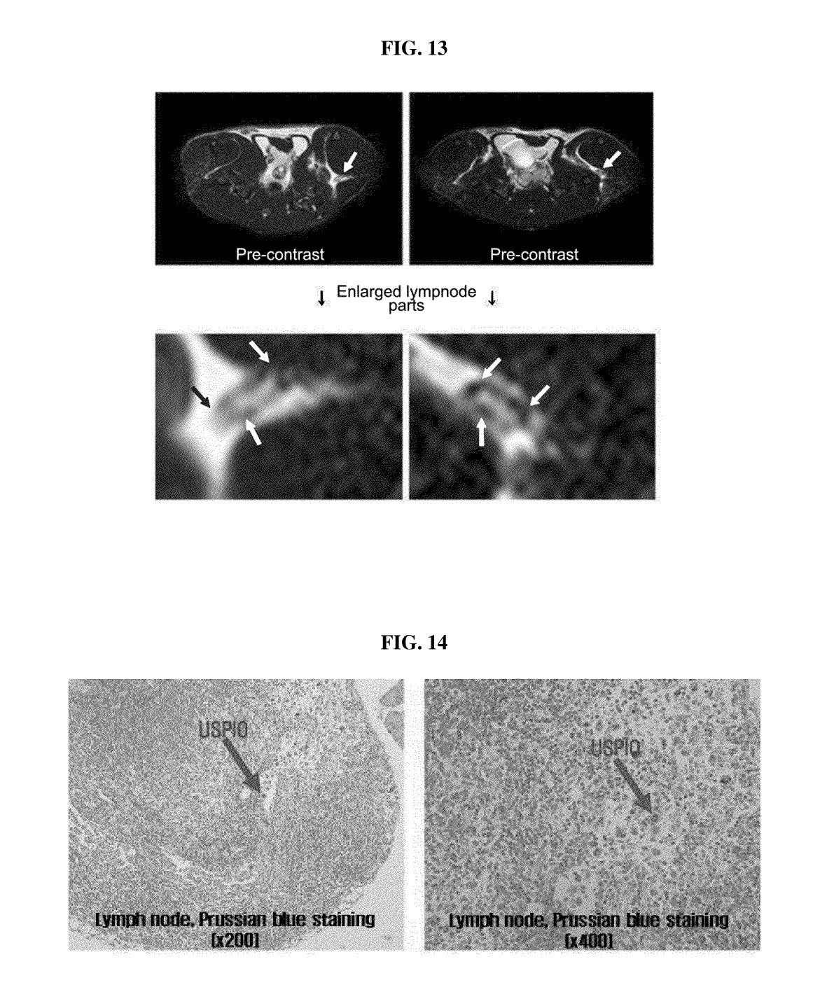

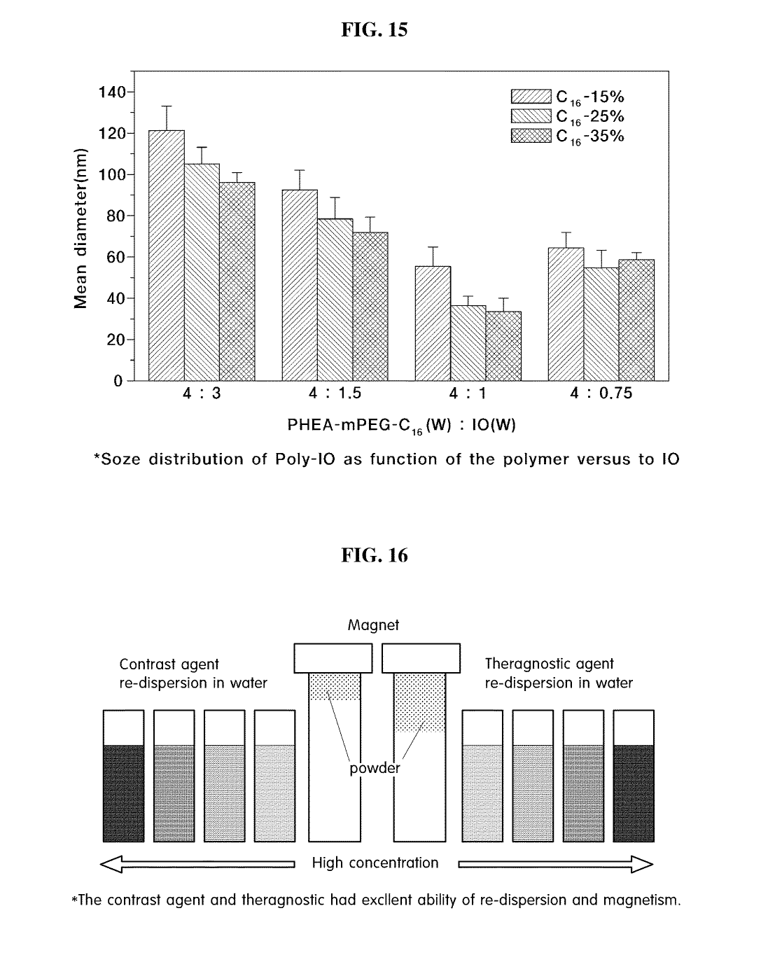

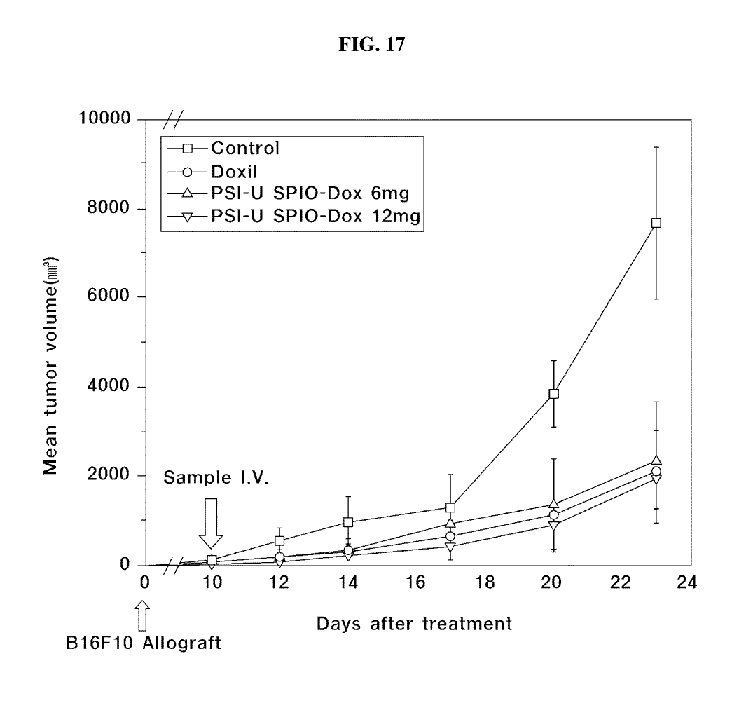

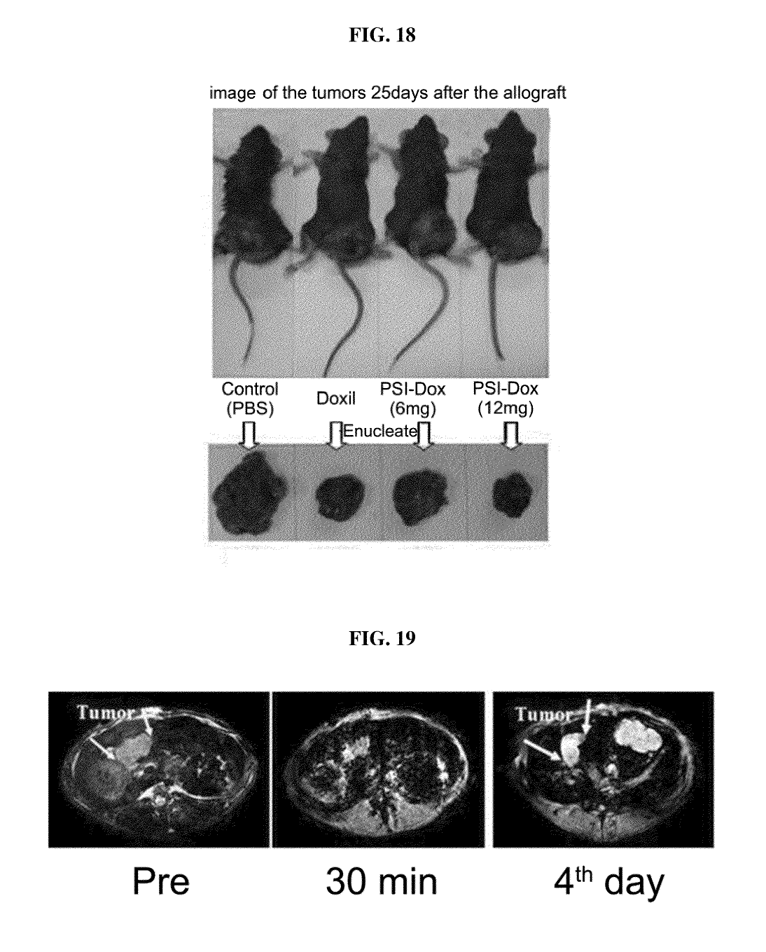

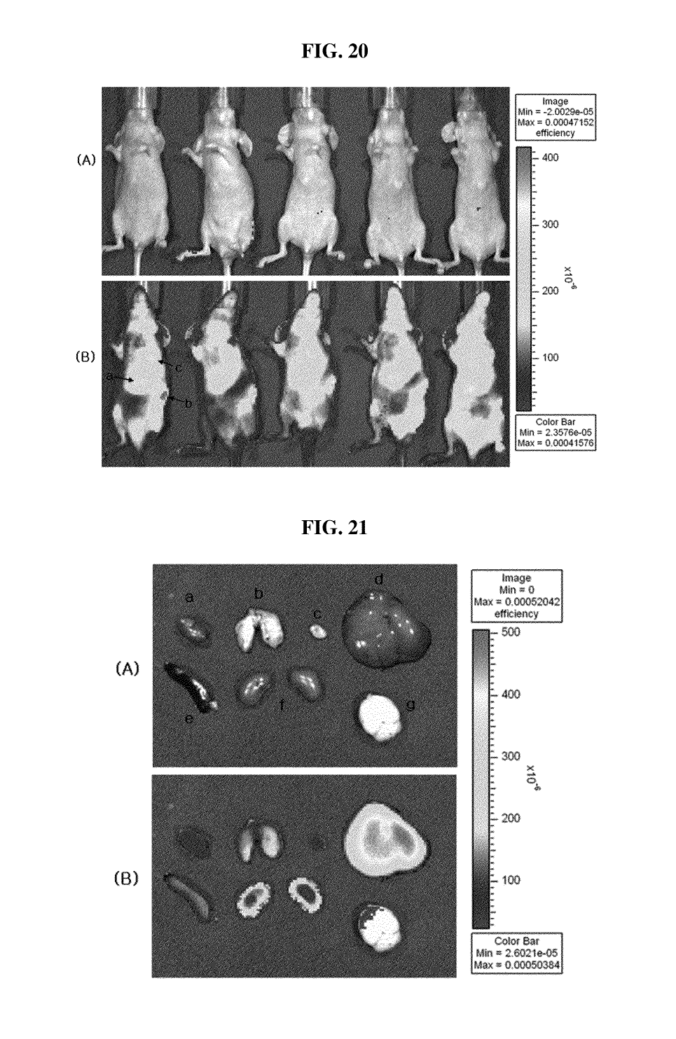

### INDUSTRIAL APPLICABILITY

From the foregoing, the polysuccinimide-based polymers introduced with a hydrophilic group and a hydrophobic group, was further introduced with an alkylenediamine group to manufacture an MRI iron oxide contrast agent or a near infrared fluorescent contrast agent. In particular, the MRI iron oxide contrast agent of the present invention was shown to have excellent bioavailability, ability to control particle size, and contrast effect.

The contrast agent of the present invention was also shown to have multifunctions and drug encapsulation ability, thus enabling to encapsulate an anticancer agent thereby manufacturing a theragnostic agent. Further, the contrast agent uses an inexpensive iron compound as a T2 contrast agent for MRI measurement and can be also manufactured via a large scale manufacturing process due to the simplicity of the process, and is expected to be widely used in medicinal fields due to its dual effects of a contrast effect and an anticancer activity.

1. A polysuccinimide-based compound comprising: (a) a main chain consisting of polysuccinimide synthesized from maleic anhydride having a molecular weight in the range of 1,000-100,000; and (b) a branch chain consisting of a hydrophilic group derived from polyethyleneglycol, polyvinylpyrrolidone, dextran, polyethyleneoxide, polylysine or polyvinylalcohol having a molecular weight in the range of 100-20,000; a hydrophobic group derived from C3-C80 amine or phospholipid; and an alkanolamine group comprising a contrast agent selected from the group consisting of gadolinium, manganese, iron oxide, aluminum, silicon, barium, yttrium and rare earth elements.

2. The polysuccinimide-based compound according to claim 1, further added with an alkylenediamine group, whose terminus is bound with a fluorescent material modified with said N-hydroxy succinimidyl ester (NHS-ester).

3. The polysuccinimide-based compound according to claim 2, wherein said fluorescent material includes carboxyfluorescein diacetate N-succinimidyl ester (carboxyfluorescein diacetate N-succinimidyl ester), CYDye™ 3.5(mono reactive NHS-ester) or CYDye™ 5.5(mono reactive NHS-ester).

4. The polysuccinimide-based compound according to claim 1, having a particle size of 20-200 nm.

5. A contrast agent comprising a polysuccinimide-based compound according to claim 1.

6. The contrast agent according to claim 5, wherein an anticancer agent is encapsulated.

7. The contrast agent according to claim 6, wherein said anticancer agent includes doxorubicin, epirubicin, docetaxel, paclitaxel, valrubicin, cisplatin or tamoxifen.

8. A method of manufacturing a multifunctional contrast agent by purifying said contrast agent of claim 5 by using a dialysis device with tangential flow separation module.

9. The method of manufacturing a multifunctional contrast agent according to claim 8, wherein said contrast agent, after purification, is dissolved in dimethylsulfoxide or dimethylformimide, and then irradiated along with an anticancer agent by using an ultrasonicator for 100 minutes or less, thereby encapsulating said anticancer agent into said contrast agent.

10. A contrast agent comprising a polysuccinimide-based compound according to claim 2.

11. A contrast agent comprising a polysuccinimide-based compound according to claim 3.

12. A contrast agent comprising a polysuccinimide-based compound according to claim 4.
